(19) United States
(12) Patent Application Publication
(10) Pub. No.: US 2016/0136213 A1
(43) Pub. Date: May 19, 2016

(54) Process to suspend cannabis oil in glycerin

(71) Applicant: Charles William Paul, Owasso, OK (US)

(72) Inventor: Charles William Paul, Owasso, OK (US)

(21) Appl. No.: 14/508,370

(22) Filed: Nov. 14, 2014

(51) Int. Cl. A61K 36/185; A24B 15/26; A61K 9/00

(52) U.S. Cl. CPC A61K 36/185; A61K 9/007; A24B 15/26

(57) ABSTRACT

The applied for method of inserting cannabis oil into glycerin represents a significant improvement over the current technology today. First, no reliable or food safe suspension mechanism exists in the medical marijuana market today. The applied for technology could be used immediately to administer the medication and also to control concentration of the medication. There are current technologies that are in use in the hemp oil and recreational markets, but none are as food safe as the applied for method.

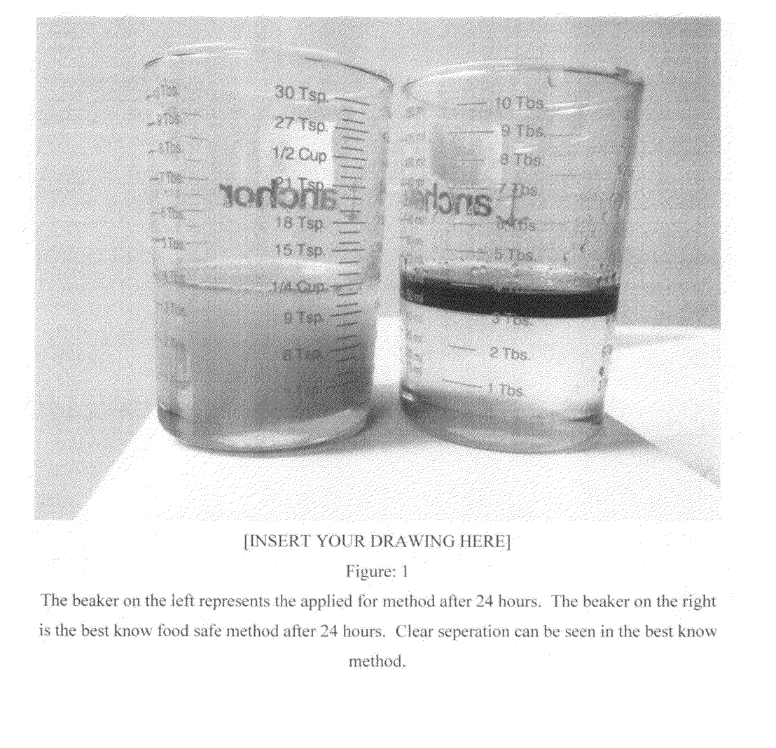

### BACKGROUND

Current methods of suspending cannabis oil in glycerin are not as effective as the applied for method. Current methods include using a combinor such as propylene glycol or PEG400 which have food safety concerns. These current methods also do not stay suspended for extended periods of time.

### INVENTION SUMMARY

This process provides an easy to perform and food safe method of inserting cannabis oil into glycerin.

### INVENTION DESCRIPTION

1. Oil preparation phase—Depending on the type of cannabis material being inserted into the glycerin the initial preparation methods of the oil would vary. The end process of this oil preparation would be a clean oil without alot of plant matter. Additionally, it would be an oil without a lot of suspended matter. Each known method is described below, but other methods exist for oil preparation and this does not preclude those methods:
A. Rendering—this process would involve use of raw plant material. The plant material is ground into small particles. The small particles are cooked in hemp oil for enough time to allow for decarboxylating. The mixture is strained and the remaining oil will be the oil used for the insertion.
B. Using extracted cannabis concentrate—the cannabis material is prepared by softening it with alcohol, hemp oil, or another liquid which would not alter the properties of the cannabis. Then the material is placed in hemp oil, or another liquid which would not alter the properties of the cannabis, and heated until is is completely dissolved in the liquid. This liquid mixture will be what is inserted into the glycerin.
C. Other methods—there are many manufactures of cannabis oils. All of these oils can be inserted into glycerin and would require then to be “clean” meaning free of any large plant matter.
2. Glycerin preparation phase—while it is not necessary, generally, the glycerin will be prepared with water and alcohol. Water and alcohol are added to the glycerin at a varying degree of total volume ratio. The water and alcohol percentage varies by the type and concentration of the oil being inserted. This solution is then mixed.
3. Insertion—the oil is prepared with a food safe soy emulsifier. The soy emulsifier must have the proper chemical structure to combine to both glycerin and cannabis molecules.
4. The oil solution is then combined with the glycerin solution and mixed aggressively. This creates the cannabis oil in glycerin suspension.

The invention produces a lasting suspension where the cannabis oil is suspended in glycerin. This allows for more accurate dosing of the cannabis oil and a better control of concentration of the final suspended product.

Use of this invention would include but not be limited to: improving current methods of suspension of cannabis oils; creating a food safe suspension for cannabis oils; creating a more reliable and accurate way to dose medical marijuana; creating a flavored cannabis oil suspension; creating an oral medical marijuana medicine which can be more accurately dosed; creation of a flavored marijuana infused juice which can be used in a traditional electronic cigarette.

### IMPROVEMENTS ON PRE-EXISTING INVENTIONS

Current methods are not food safe and do not remain in suspension for extended periods of time.

### OTHER USES OR APPLICATIONS FOR THIS INVENTION

This invention, when used with completely legal throughout the US hemp oil, could create a better base mixture for electronic cigarette e-liquid and could lead to an elimination of propylene glycol which is prevalent in the electronic cigarette industry. Additionally this invention provides a method to dose medical marijuana which does not exist today. Further, the oil/glycerin suspension can be flavored with food flavorings to make an oral medication or a medication which can be used in traditional electronic cigarette hardware.

1. improves current methods of suspension of cannabis oils;

2. creates a food safe suspension for cannabis oils;

3. creates a more reliable and accurate way to dose medical marijuana;

4. creates a flavored cannabis oil suspension which can be taken orally or used in electronic cigarette hardware;

5. creates an oral medical marijuana medicine which can be more accurately dosed;

6. creates an oral medical marijuana medicine which can be flavored and used in traditional electronic cigarettes;

7. creates of a flavored marijuana infused juice which can be used in a traditional electronic cigarette.
